(19) United States
(12) Patent Application Publication
(10) Pub. No.: US 2011/0015538 A1
(43) Pub. Date: Jan. 20, 2011

(54) SYSTEM AND METHOD FOR ANALYZING ELECTROENCEPHALOGRAPHY DATA

(76) Inventor: Thomas Virgil Matthews, JR., Topeka, KS (US)

Correspondence Address: SPENCER, FANE, BRITT & BROWNE, 1000 WALNUT STREET, SUITE 1400, KANSAS CITY, MO, 64106-2140, US

(21) Appl. No.: 12/505,033

(22) Filed: Jul. 17, 2009

(51) Int. Cl. A61B 5/0476

(52) U.S. Cl. 600/544

(57) ABSTRACT

A system and method for analyzing EEG data. The system comprises data from at least one or all of the 19 (or more) standard scalp locations, a computing device 100 operable to calculate deviance for the data, and a single score for each of a plurality of scalp sites based on the deviance. An output device 10 presents a ranked list of the single scores to identify sites showing abnormal EEG, or a single score for all sites and pairs combined that may be used in assessment or operant conditioning.

### FIELD OF THE INVENTION

The present invention relates to analyzing electroencephalography (EEG) data. More particularly, the invention relates to computer programs that receive EEG data, perform a series of transformations with the EEG data, produce a single score for each of one to nineteen or more scalp sites, produce a single score for each of the scalp site-pairs, produce a single combined score for all the measured sites and site-pairs, and present the data in a readily understandable format, which can be quickly assimilated.

### BACKGROUND OF THE INVENTION

EEG refers to the recording of the brain's spontaneous electrical activity, generally from multiple electrodes placed on the scalp. In the clinical neurology setting, a health professional with specific training in the interpretation of EEGs reads the EEG results by visually inspecting the waveforms, searching for momentary transient activity that indicates abnormality. Computer signal processing of the EEG, or quantitative EEG (qEEG), is in wider use including brain research, anesthesia monitoring, medication selection and testing, verification of brain injury, and various brain rehabilitation procedures including neurofeedback. In qEEG, the electrical activity is sampled and recorded as numbers that can then be analyzed and converted into a display of brain functioning. The numbers convey information about electrical activity at sites, or site-pairs, where the data was measured. Several qEEG techniques include power-spectral analysis, evaluation of asymmetries, computation of coherence and phase-delay, tabular and topographic displays of those and other measures, and statistical comparisons to normative values (z-scores).

Neurofeedback (NFB), sometimes called EEG biofeedback, is a technique that presents a client with real-time feedback of their brainwave activity, typically in the form of an audio-video display. Based on operant conditioning, it may or may not involve the client forming a conscious understanding of brain activity during the training process. The goal is to transform the patterns of electrophysiological activity under the sensors so as to achieve an enduring alteration and improvement. Thus, positive feedback is given when EEG activity is appropriate for the training goal, and the positive feedback is withdrawn when the brain activity is outside the acceptable range for the training. Applications where neurofeedback has been researched include treatment of substance abuse, anxiety spectrum disorders, affect disorders, migraine, seizure, behavior disorders, and diverse cognitive deficits, among other applications.

qEEG and NFB have similarities and differences. Each involves the collection and analysis of EEG data, that is, measuring voltage at some or all of the 19 (or more) standard scalp locations. These locations are defined by the “10-20 System”, an international standard of EEG electrode placement. Both qEEG and NFB may be enhanced by the use of z-scores. A z-score is a measure of how normal or abnormal a measurement is in comparison to a value in a file, or database, containing expected scores. Although qEEG is a broader term, in the discussion that follows qEEG is used in the narrower sense of a comparison of EEG to the scores of (generally symptom-free) people in a database.

The primary difference between qEEG and NFB is that qEEG is an assessment procedure in which EEG data is retained for later analysis, whereas NFB is a treatment procedure in which EEG data is used in real-time. NFB uses the data immediately and continuously to conduct real-time training. Because of the need for immediate data, the data is not manually edited, and in many cases the data collection criteria are somewhat relaxed due to the realities of daily business practices and intended use.

There is currently no system that quickly and efficiently determines the most abnormal brain region from EEG. Previous systems are unable to summarize EEG data in an acceptable manner for this purpose as certain EEG input-sets are thought to be unsuitable for combination.

### SUMMARY OF THE INVENTION

The present invention solves the above-described problems and provides a distinct advance in the art of analyzing EEG data. More particularly, the present invention receives as input the output of EEG acquisition software converted to z-score tables, conducts a set of transformations, and provides a summarized report of static or real-time EEG data, depending upon the application, in a readily understandable format, which is quickly assimilated.

The present invention accepts as input a relatively large number of variables (over 5000 in some instances), converts them into a simplified and optimized set of scores which it then sorts and displays so as to address specific needs of the user based on expert knowledge of the application for which the output is to be used.

In one embodiment, the present invention is implemented with a computer program stored on a computer-readable media for directing operation of a computer. In one embodiment, the computer program receives static files previously saved by a system that collected and analyzed EEG data. In another embodiment, the computer program includes EEG acquisition capabilities. In yet another embodiment, the computer program communicates with a system collecting EEG data. In each embodiment, the computer program receives z-score tables or data stream as input, transforms the z-scores or data stream into a summarized report by calculating deviance scores for single-sites and site-pairs, converts the deviance scores into a single score for each of one to nineteen or more scalp sites, produces a single score for each of the scalp site-pairs, and displays a ranked report based on the single scores identifying probable defective sites and pairs.

In one embodiment, the present invention receives qEEG spectral-analysis results that have been converted to z-scores, converts the z-scores to deviance scores, and computes hit rates, means and other summary measures (median, standard deviation) of appropriate output sets of scores. In one embodiment, the present invention attributes to each of the two sites comprising a pair the z-scores for that pair with respect to the variables of interest. The deviance is obtained by computing the absolute value of each z-score and a mean is calculated for all z-scores associated with a given site. In another embodiment, the sum of absolute values is calculated for all z-scores associated with a given site. In one embodiment, a ranked list is presented on a display, giving the user in a readily understandable format, which can be quickly assimilated, a report with the most deviant sites, site-pairs, and other combinations of sites, based on combining all, or various subsets of, the spectral-analysis z-score findings. This information output is particularly useful because the most common purpose of qEEG and the resulting z-scores is to identify the scalp sites and interrelationships that are most abnormal. Achieving this identification manually with qEEG is a difficult and time-consuming process, and the present invention provides a more accurate solution that can be provided with either static or real-time data.

In one embodiment, the present invention differentiates the outputs of various montages, described below, and handles them properly. It is also common to collect multiple data sets in one assessment, to address reliability and validity, often using data collected under different conditions (eyes-open and eyes-closed are the most common, and databases have both comparisons available). The present invention, in one embodiment, combines input from multiple analyses to produce a single report.

In one embodiment, the present invention is used as a planning or assessment tool whereby the user inputs the results of previous qEEG analysis. In another embodiment, the present invention is used to monitor real-time EEG data for monitoring and training purposes.

In one embodiment, the present invention is integrated with billing and records-management software. In one embodiment, the single scores are used to measure the effects of medication. In another embodiment, the single scores are used to measure treatment response.

These and other features of the present invention are described in greater detail below in the section titled DETAILED DESCRIPTION OF THE INVENTION.

### BRIEF DESCRIPTION OF THE DRAWING FIGURES

The present invention is described herein with reference to the following drawing figures, with greater emphasis being placed on clarity rather than scale:

FIG. 1 is a diagram representing the data flow of EEG gathering and analysis that occurs before using the Planner embodiment of the present invention;

FIG. 2 is a diagram representing the data flow of the Planner embodiment of the present invention;

FIG. 3 is a diagram representing the data flow of the feedback loop and control in neurofeedback;

FIG. 4 is a diagram representing the data flow of the Monitor embodiment of the present invention;

FIG. 5 is a network diagram representing an alternative embodiment of the Monitor, integrated with billing and record management software;

FIG. 6 is a network diagram representing the Trainer embodiment of the present invention;

FIG. 7 is a network diagram representing an alternative embodiment of the Trainer implemented as a single-user or remote use device.

### DETAILED DESCRIPTION OF THE INVENTION

The electrical activity of the brain can be measured and studied by the potentials that the EEG records from the scalp. Neurons are electrically active cells and responsible for creating action potentials. Action potentials are discrete electrochemical signals that execute the brain's functions. These signals travel down axons and cause the release of chemical neurotransmitters at an area of contact between adjacent neurons, known as the synapse. Neurotransmitters fit into a receptor in the dendrite or body of a neuron on the opposing side of the synapse known as the post-synaptic neuron. An altered tendency to fire is created within the post-synaptic neuron when the neurotransmitter is combined with the receptor, either inhibiting or exciting the neuron to fire. When a neuron receives a preponderance of excitation, the post-synaptic currents created in the thousands of dendrites of a single neuron generate an action potential which then allows the neuron to pass the electrochemical signal on to other neurons. When thousands of neurons radially oriented to the scalp fire simultaneously, surface EEG measurements are possible.

EEG recording is typically obtained by placing electrodes on the scalp with a conductive gel or paste. Commercially available surface cleaners and abrasive preparations, such as Nuprep or Redux, are used to properly prepare the skin surface and achieve sufficiently low impedance to enhance flow of the electrical signal to the electrodes. Nineteen recording electrodes or sites are used in most clinical applications; however, a smaller or larger number may be used depending on the type of recording being performed.

Each EEG signal represents a difference between the voltages in two circuits, arranged among three electrodes (the “active”, reference, and a common ground). This arrangement takes advantage of common-mode rejection, such that all electrical signal common to the two circuits is cancelled out and the majority of potential artifact is thereby removed.

EEG data is gathered as samples of common-mode-rejection voltage, sampled initially at 2000 to 4000 samples per second. This high sampling rate is to facilitate visual inspection for transient events, and for qEEG the data is then down-sampled to 100 to 200 samples per second. This record is manually edited so that only artifact-free data epochs remain, and the resulting dataset is re-assembled, commonly by use of overlapping windowing for subsequent analysis. A power spectral analysis is conducted, which yields the power or magnitude of the voltage component attributed to each frequency range used for the analysis. For example, if the analysis inquires for the traditional five frequency ranges at the conventional 19 scalp locations, the resulting table shows a 5×19 table of the voltage in microvolts for each frequency measured at each site. This is the fundamental data and the remaining variables are computed from it, including amplitude asymmetry, coherence, phase delay, and additional variables. At this stage the information is descriptive of the data obtained, but no conclusion can be made regarding potential abnormality of the observations.

Examples of Food and Drug Administration (FDA)-compliant, stratified z-score databases include the NxLink, NeuroGuide, and Brain Resource International databases. Comparison of EEG power spectral findings to such a database yields z-scores, a measure of how abnormal a finding is in comparison to the population, and from which one may find statistical measures that indicate whether the finding is either likely to be a chance variation, or is statistically significant.

The statistical process sets limits, so that if the finding has a z-score larger than the limit, the finding is significant. If the finding is larger than the next more-stringent limit, it is more significant, which translates to it being less likely to be seen just by chance. Z-scores can be positive or negative, indicating a finding greater or smaller than expected. A z-score of zero indicates that the finding is exactly the number found to be the population average.

When a customary 19 channel full-head EEG is submitted to a database and the usual variables tested, one obtains several thousand z-scores. Those are provided in tables, but more easily understood when displayed in topographic map-like images. The result is a very large amount of highly detailed information about localized degree of abnormality, which is profoundly useful for many purposes. However, users generally perform qEEG to answer certain key questions. The present invention provides post-analysis transformations of this large z-score file, emphasizing precision answers for those key questions.

The EEG recording may be set up in a variety of ways referred to as montages. A Bipolar montage displays the difference between two adjacent electrodes, whereas a Referential montage displays the difference between each of the scalp electrodes and a designated reference electrode. In a Linked Ears Reference montage, the two earlobes are electrically linked or digitally averaged and the resulting value is used as the reference for each scalp electrode. The Average Reference montage uses as a common reference an average of all 19 scalp electrode signals. Similarly, the Laplacian montage uses the weighted average of nearby electrodes as the reference. Because each montage has its strengths and weaknesses, a thorough evaluation involves analysis of more than one montage. The different analyses produce mostly, but not entirely, similar output z-score tables.

qEEG mapping requires colleting discrete sets of data under rigorously defined conditions so that the data is strictly comparable to a database of expected results. A series of highly reliable datasets are obtained for descriptive, diagnostic, or planning purposes. These datasets are then retained for review and thorough analysis at a later time. The data is edited to remove artifact, is compared to the database of expected results, and a series of analyses are conducted that produce z-scores. The results of the analysis are customarily displayed as charts and topographic representations of the z-scores, typically requiring many pages. A trained professional then issues a formal, written report of findings and interpretation. Finally, the scores themselves may be exported for additional post-analysis by the trained professional. qEEG is employed in many endeavors including localization of brain abnormality, measurement of medication effects, computer modeling of brain function, basic brain research, and treatment planning for several procedures including neurofeedback.

Generally, in order to conduct appropriate training for a client's needs, NFB is designed based on needs assessment, correlation of needs with cognitive function, published research on brain localization of function, and the client's qEEG findings that localize the actual brain function deficits. Given this set of information the user designs a highly individualized treatment plan for the client.

There are other assessment schemes used to select scalp sites and parameters to use for NFB, or more precisely, to predict what brain regions are likely to be abnormal. The most common is simply to rely on symptom report. There are several computerized examples of such symptom-to-site assessment schemes. These systems start with symptom inquiry, rely on the research literature of brain functional-localization, and produce a list of sites likely to be associated with the symptoms. The result is mere conjecture about the client's actual brain functional deficits, and to have suitable validity, should be correlated with qEEG findings.

The generally accepted procedure in qEEG interpretation is to develop hypotheses based on symptoms, and then to test those hypotheses with the qEEG. The statistics of qEEG analyses are based on and require this procedure. There is currently no qEEG analysis system that explicitly implements hypothesis testing. Interpreting qEEG is time consuming and requires professional expertise to visually examine many pages of output. Users generally want to know what parts of the brain are most seriously dysfunctional. There is currently no qEEG analysis system that quickly and efficiently combines all z-score findings to determine the overall most abnormal brain regions.

Recent advances in NFB allow the EEG voltages to be compared to an appropriate database in real-time so that z-scores can now be used for training purposes. Whether the EEG voltages or derived z-scores are used for NFB, the continuous flow of information is compared to a second set of “training” criteria designed to shift brain function by operant conditioning. In this manner, continuous feedback about success in meeting those training criteria is displayed to the client in some way that rewards success.

Most NFB systems involve significant compromises in the integrity of the data supplied to the user for monitoring progress and outcomes, due to confounding of training criteria with outcome measures. Training criteria customarily are used to shape, and for this reason are repeatedly adjusted during a training session. Consequently a measure of how often the training criterion is met becomes a purely relative measure and meaningless as an outcome measure. This is not to say that current systems offer invalid output. They offer trend lines and tables, but no summary measures that can be quickly and easily assimilated. As a consequence, most users simply do not record output data files because the limited benefit does not justify the time and expense required.

In addition, for many treatment procedures including NFB, it is important for the user (often a clinician) to track a client's progress during and across sessions in order to properly guide treatment. A typical z-score NFB system produces a huge data stream, with 248 variables and 200 samples per second, just for a 4-channel system. As a consequence of this data volume and of the measurement-confound just described, no solution exists that provides meaningful summary information about progress, on either a real-time or intermittent basis. Users today merely track progress by relying on symptom-report alone, a notoriously unreliable measure.

Clients, family members, and insurers are generally interested in progress, particularly when payments are at issue. The insurer may demand evidence of treatment response and improvement, and cease paying when progress stops. The trend toward such pay-for-performance in medical care is advancing rapidly. NFB is imminently quantitative but the pervasive failure to capture and summarize the session data has nearly squandered NFB's potential capacity to document progress.

The current approaches to utilizing qEEG and EEG NFB data all produce a significant amount of detail that must be analyzed by a trained professional, a process that can be quite time-consuming and expensive and is not practical for many purposes where EEG can provide a significant advantage, such as for use in monitoring NFB, measuring treatment response, or integration with billing and digital medical records systems. For example, z-score NFB customarily is accomplished using two, four, or nineteen sites simultaneously. The number of variables mounts rapidly because of the interactions between sites, and even a four site system includes 248 variables. Thus, a common display in such a four site system results in 248 z-scores presented at once, changing approximately two times a second (and that is just the display refresh rate). Previous systems are unable to summarize EEG data as means (or other measures) of deviance scores because certain input-sets are thought to be unsuitable for combination. These input sets include scores for individual scalp sites as well as scores for site-pairs. As a result, there is currently no system or method for transforming EEG data into a readily understandable summary format, which can be quickly assimilated. Many users have attempted to address this need by using makeshift solutions that are inaccurate, cumbersome, and impractical.

With reference to the drawing figures, a system and method is herein described, shown, and otherwise disclosed in accordance with various embodiments, including a preferred embodiment, of the present invention. Broadly, the present invention provides a system and method for analyzing, summarizing and presenting EEG data.

The present invention can be implemented in hardware, software, firmware, or a combination thereof. The computer may be any computing device such as an IBM compatible personal computer including those manufactured and sold by Dell, Compaq, Gateway, or any other computer manufacturer.

The computer program of the present invention is stored in or on computer-readable medium residing on or accessible by the computer for instructing the computer and other components of the equipment to operate as described herein. The computer program may run in DOS, Windows, or any other operating system environment and, in one embodiment, comprises an ordered listing of executable instructions for implementing logical functions in the computer and any computing devices coupled with the computer.

The computer program can be embodied in any computer-readable medium for use by or in connection with an instruction execution system, apparatus, or device, such as a computer-based system, processor-containing system, or other system that can fetch the instructions from the instruction execution system, apparatus, or device, and execute the instructions. In the context of this application, a “computer-readable medium” can be any means that can contain, store, communicate, propagate or transport the program for use by or in connection with the instruction execution system, apparatus, or device. The computer-readable medium can be, for example, but not limited to, an electronic, magnetic, optical, electro-magnetic, infrared, or semiconductor system, apparatus, device, or propagation medium. More specific, although not inclusive, examples of the computer-readable medium would include the following: an electrical connection having one or more wires, a portable computer diskette, a random access memory (RAM), a read-only memory (ROM), an erasable, programmable, read-only memory (EPROM or Flash memory), an optical fiber, and a portable compact disk read-only memory (CDROM). The computer-readable medium could even be paper or another suitable medium upon which the program is printed, as the program can be electronically captured, via for instance, optical scanning of the paper or other medium, then compiled, interpreted, or otherwise processed in a suitable manner, if necessary, and then stored in a computer memory.

The computer program initially displays a main menu or screen on the control display. The main menu provides links to various EEG analysis functions. The main menu also provides links to sub-menus such as setup whereby a user may configure various options and flags to their specific needs and desires. In one embodiment the user is required to select sites of interest comprising hypotheses to be tested, and to select sites likely to show artifactual data due to any underlying skull defect. In one embodiment a symptom-to-location on-screen questionnaire is included, allowing the system to provide an integrated qEEG hypothesis generation and testing tool.

Because EEG NFB systems lack adequately useful capture of summary output data measures, they currently are not viewed as research or diagnostic platforms for procedures other than NFB. However, because the present invention provides improved summary real-time analysis of z-score EEG, it allows EEG NFB systems to effectively compete with functional magnetic resonance imaging (fMRI) and single photon emission computed tomography (SPECT) to provide real time functional analysis of brain activity. Although EEG does not provide precise localization or anatomical imaging, it significantly outperforms fMRI and SPECT with respect to time-resolution, cost, and measuring of actual, rather than imputed, brain physiology.

Several embodiments of the invention employ broadly the same EEG transformations and summary measures to 1) summarize a qEEG so as to assist the user in its interpretation and treatment planning (the Planner), or 2) monitor real-time EEG in various applications such as NFB, rTMS, or brain-computer interfacing (the Monitor), or 3) implement the summary measures as a training criterion in NFB (the Trainer).

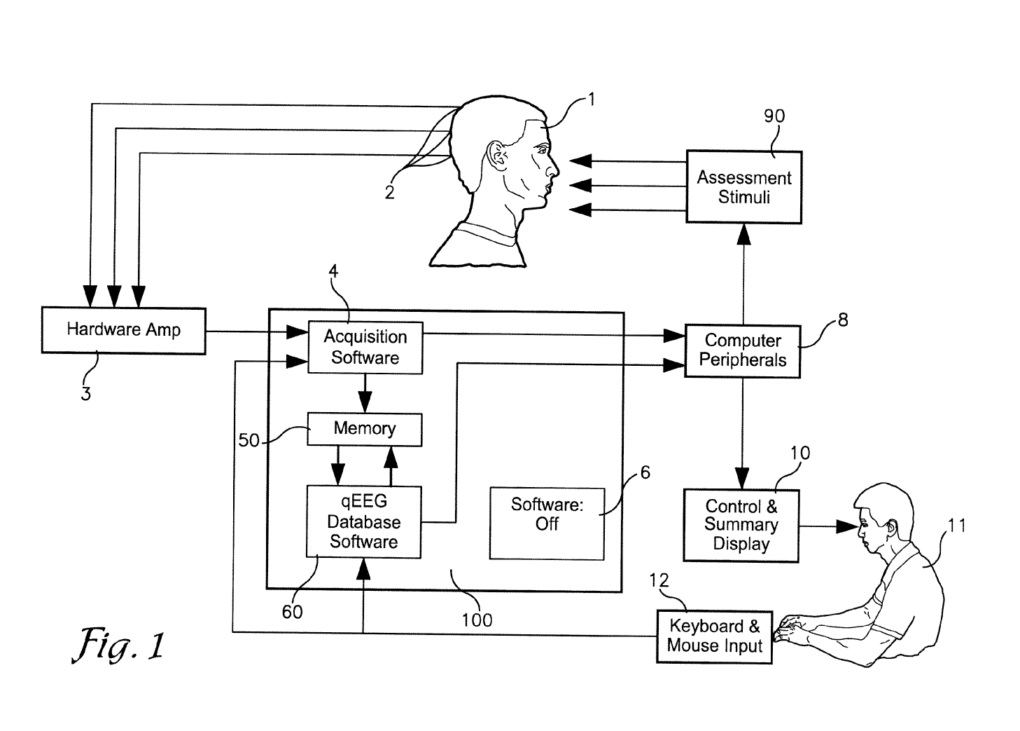

Referring to FIG. 1, the context of the Planner embodiment of the invention may be viewed by examining those assessment operations which would customarily occur before the Planner is used. Here, in one embodiment the invention software 6 is off. During EEG acquisition, the client 1 produces brain electrophysiology detected by sensors 2 and conveyed by wires to the hardware amplifier 3. The amplifier together with the acquisition software 4 samples and stores EEG data in memory 50, provides operational information to a control display 10, and allows the user 11 to make keyboard and mouse input 12. During analysis of the EEG generally the data has been stored in memory 50. The user 11 selects EEG data files from memory 50 and using the qEEG database software 60 performs the manual editing, and conducts analyses that produce z-score files saved for later use.

In another embodiment, the invention includes EEG acquisition software. The computer program and equipment illustrated and described herein are merely examples of a program and equipment that may be used to implement the present invention.

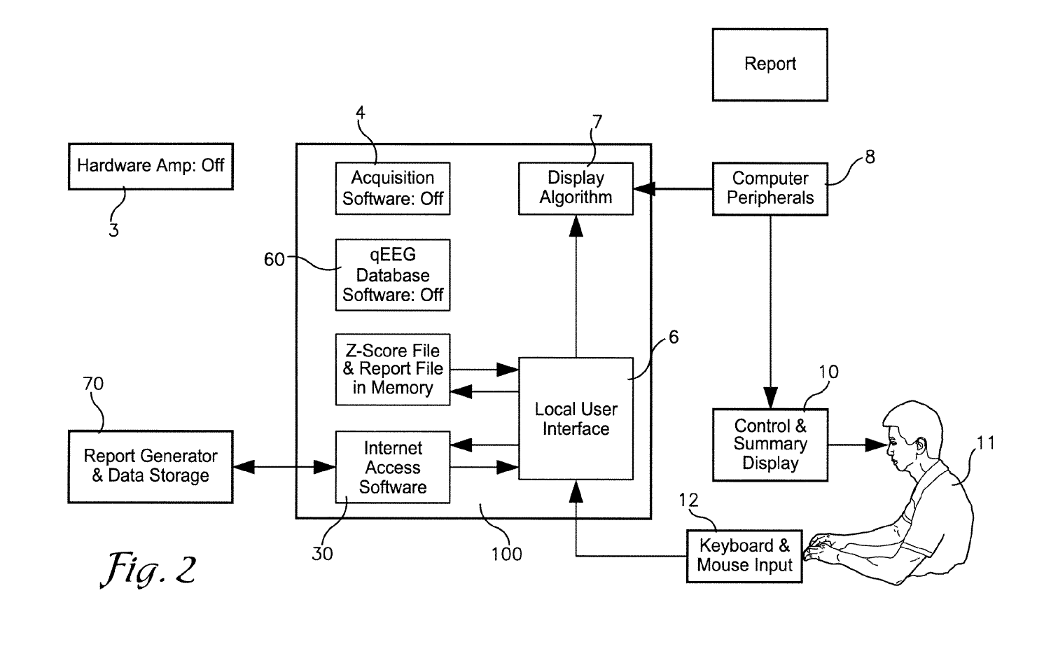

Referring to FIG. 2, in one embodiment the Planner consists of an online report generator and database. In one embodiment, the Planner resides on the local computer. In another embodiment, the Planner includes a local user interface as illustrated. User 11 accesses the data file 1 from memory, provides additional keyboard or mouse 12 input of information required by the invention software submits the request for a report via the Internet 30 to the online system 70, whereby a user interface 6 may save or display 10 the report. In one embodiment, the Planner report is a text display and in another it is a three dimensional figure of a head or brain with the data interpolated and displayed so that the user may interactively adjust parameters and view the results.

The set of all qEEG z-score variables may contain subsets of variables, such as the frequency-bin variables, not all of which are mutually orthogonal. In one embodiment of the invention, a subset is selected of orthogonal variables which may or may not contain the full range of the underlying measure, such as EEG frequency. Operating with that subset, the absolute-value is taken of each member and a further selection is made of all subset member absolute-values that meet or exceed a chosen threshold; these cells may be termed “hits”. A count is made of the hits, and for one or more variables the hit absolute-values are averaged. If for more than one variable then the average may or may not be computed across variables. In one embodiment the hit-count and absolute-values-average are reported for each scalp site and each site-pair, or for the site and pair subsets that exceed threshold.

In one embodiment the chosen threshold may be fixed as a level of significance such as the 95% confidence cutoff at [Z]=1.96. In one embodiment the chosen threshold may be set by the user. In one embodiment the chosen threshold may be set by the user along a continuous range interactively by means of adjusting a slider-bar or other means, and viewing the output as it changes in response to threshold adjustment.

In one embodiment the present invention runs in a client-server environment. The client application interfaces solely with the operating system of the desktop computer on which it resides and the server component on the server. In another embodiment, the present invention runs as a standalone application on a user's desktop computer. In another embodiment, the present invention is utilized online, as a software-as-a-service model, in which a server-resident application manages data file input, option selections, upload, and retrieval of completed reports. In addition, the application receives and stores online the z-score tables and raw EEG files previously created by third-party programs.

The present invention detects and reports any mismatches between the data received and information provided by the user in order to assure valid output, and meet various FDA compliance requirements. In addition, the present invention supports sale of services, allows that it interface with credit-card clearing, and download usage and financial information. Although in the Planner embodiment, there is no direct communication with other EEG software, it recognizes and correctly handles data formatted and saved using various EEG acquisition software.

The present invention allows the user to properly identify the dataset with respect to analysis specifications, report labeling, and a unique dataset identifier number capable of associating repeat data and reports for a given client.

Again referring to FIG. 2, the Planner embodiment receives one or more static z-score tables from which it produces a static report or set of reports. A user interface is provided that allows the user to browse to and select the desired data file(s), and then for each, specify the collection condition, the analysis montage, the source program, a unique client identifier number, a client identification field that is not permanently stored, and the report(s) requested.

The skull attenuates the magnitude of the EEG about one hundred fold. A problem often faced by those conducting EEG analysis is that if there is a breach in the skull, for that reason alone the signal of normal tissue below will appear abnormal when compared to a qEEG database comprised of intact-skull EEGs. People who survive serious brain damage events often have traumatic or surgical skull deficits that do not close. These people are likely clients for qEEG, NFB and similar procedures. This presents a significant problem unaddressed by any existing system. The present invention requires the user to identify what sites, if any, are over skull deficits, and then offers the user options for adjusting to those issues. If the user declines to specify such information, a warning is provided. The user also indicates scalp sites of interest based on the user's expectation of importance for the intended use of the analysis output. For example a clinical user may know symptoms, from which he expects that certain scalp sites will be important in remediation. Again, if the user declines to specify such information, a warning is provided because the statistics of qEEG are based on testing hypotheses.

In one embodiment, after the user inputs the requested information and initiates analysis, the Planner associates the information with the user's unique user identifier, compares the data table parameters and file-extension with the supplied analysis-montage information to verify compatibility with the expected data-set size and confirm completeness. The system uploads the user-designated file, initiates analysis, and generates and downloads the report.

In one embodiment, to perform the analysis, the Planner accepts into an array the classes of variables found in the z-score tables, and stores the variables first in the order presented and then in the following descending order: site, major variable class (power, attributed asymmetry, attributed coherence, attributed phase-delay or other variables), and frequency band. Only frequency bands that are orthogonal are selected for future analysis. The array is populated with the z-score values and the array and the original z-score file are stored in the database. The present invention is designed to accept input from several qEEG analysis programs and produce reports combining those inputs. It is also designed to accept new EEG variable classes when they enter common use.

The absolute value is computed for each z-score value in the array. These values are stored as a deviance score in a second array formatted like the first. Of the current four major variable classes, Power is composed of values for the 19 individual sites, whereas the Asymmetry, Coherence, and Phase are composed of values for the 171 site-pairs (all the pairing permutations). The second array format stores the variables as they are arranged in the incoming z-score table, but also provides that the deviance score for each pair is associated with each of the two sites comprising each site-pair. For example, if the pair F1-F2 has a coherence deviance score of 1.2345, then that deviance is attributed to F1 and F2, for use in further analysis, in addition to any deviance score the F1 and F2 site receives individually. The Linked Ears montage analysis provides all four major class variables, whereas Average Reference and Laplacian montage analyses provide only power and asymmetry; however, the present invention computes deviance appropriately for each montage and accommodates reports combining output across analyses for different states and montages.

The Planner further allows the user to specify a multiplicative weighting constant for each value in the array that is changed to accomplish various objectives. In one embodiment, the weight can take the values of either 0 or 0.1-1.0. This provides a method to change weighting constants individually or by class in order to include or exclude specific sites or values from analysis, or to weight the contribution of each value. The Planner also allows the user to specify an additive weighting constant for each Power value in the array, which may be changed to make phenotype corrections to computations of deviance in power.

A mean across frequency bins for each major variable class (power, asymmetry, coherence, phase, or other new variable class) is calculated by the system, including any weight factors. For the site-pairs found in asymmetry, coherence, phase, and other new variable classes, the system attributes to each of the component sites, the power deviance score calculated for the site-pair. Next, the system computes a mean of means for each site. If one of the component factors is zero due to the value of the weighting constant, then the denominator is reduced by one. The Planner then ranks the sites in order, from high to low mean deviance, and the user is able to quickly identify which site is most abnormal. In one embodiment, the same ranking is compiled for the sites with respect to coherence and with respect to phase. In one embodiment, the system ranks the pairs in order, from high to low mean deviance, and the user is able to quickly identify which pair is most abnormal. NFB training may be conducted on four sites simultaneously. In one embodiment, in order to assist with selection of 4-site combinations, the ranking of pairs is examined and those individual sites that occur in highest-ranked pairs are assembled into 4-site combinations. In one embodiment, in the output of these rankings the previous user selections of breach-sites and sites of interest are indicated.

In the Planner embodiment, or another embodiment discussed in detail below (the Monitor), the standard deviation is obtained among 1) site deviances or pair deviances as appropriate for each variable class, and 2) among overall site deviances, and 3) among overall pair deviances. In one embodiment, multiples of the appropriate standard deviation (2 SD, 3 SD, etc.) are applied as cutoff indicators of significance in the rank list of sites (or pairs) with respect to that variable.

The standard deviation is computed as follows: for simplicity, take an example with 8 electrode sites (rather than the customary 19). Assume we obtain the following deviance scores for those sites:

2, 4, 4, 4, 5, 5, 7, 9.

The eight data points have a mean (or average) value of 5. To calculate the standard deviation, we compute the difference of each data point from the mean, and square the result:

(2−5)²=3²=9(5−5)²=0²=0

(4−5)²=1²=1(5−5)²=0²=0

(4−5)²=1²=1(7−5)²=2²=4

(4−5)²=1²=1(9−5)²=4²=16

Next we average these values and take the square root. In one embodiment, any site can be suppressed with a 0 weight, and the denominator under the radical should be reduced by 1 for each site suppressed. This gives the standard deviation:

Sqrt((9+1+1+1+0+0+4+16)/8)=√4=2

Therefore, the deviances at the 8 sites show a standard deviation of 2. The value 9 is two standard deviations above the mean of 5. In one embodiment, the report of the Planner or Monitor displays the standard deviation for each ranking, and also marks within the rank list the cutoffs above each standard deviation. If the scores in the above example were deviance scores, they would be marked as follows:

“9>2SD, 7, 5, 5, 4, 2”, or by foot note, or some similar marking scheme within the list.

The Planner stores all the z-scores, then creates and stores deviance scores and rankings in a database, and identifies “sites of interest” and “breach sites”. In one embodiment, the system identifies the “sites of interest” with underline and “breach sites” with strikethrough in the report output. Personally identifying information provided by the user is included in such reports printable by the user, but is not stored in the database, a solution that simplifies HIPAA compliance.

The Planner is able to combine results from multiple analyses based on multiple z-score tables provided by the user. In one embodiment, results are combined in one automated, computed report for multiple montages of the same raw data. In one embodiment, results are combined in one automated, computed report for both eyes-open and eyes-closed data. In one embodiment, results are combined in one automated, computed report to compare pre- and post-assessment of a client.

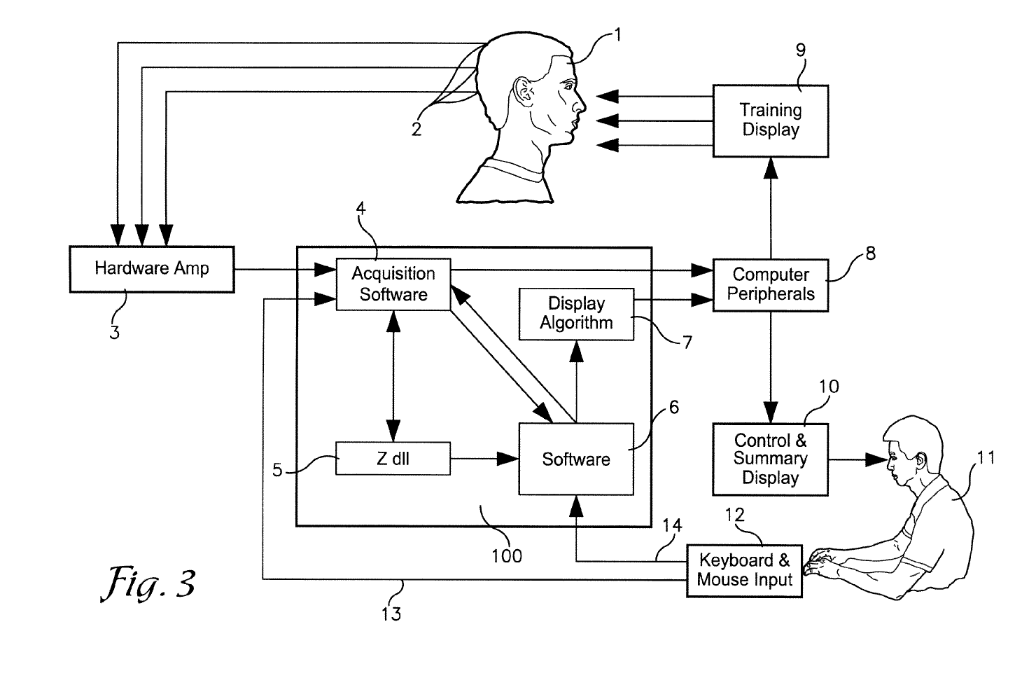

Referring to FIG. 3, the context of the Monitor and Trainer embodiments of the invention may be viewed by examining those real-time EEG operations which would customarily occur, employing NFB as an example in the case of the Monitor. In addition to the invention software, FIG. 3 shows the use of any suitable hardware EEG amplifier, any acquisition software to obtain continuous EEG data, and any real-time qEEG database software (termed the “z-score lookup software”) to obtain z-scores from that data. In another embodiment, the invention includes EEG acquisition software. FIG. 3 shows the system operation of three operational schemes: conventional NFB, z-score NFB, and z-score NFB enhanced by the invention. There are two external information loops in this system, one involving the client and one involving the user.

In each of the three operational schemes, the client 1 is continuously producing covert physiology including EEG, which can be detected as signal by equipment. Signal detection is accomplished by sensors 2, usually comprised of wires and gel-medium on the skin, and is directed to the hardware amplifier 3. The amplifier transmits a suitable raw digital data-stream to the acquisition software 4 running on the computer or in some configurations running partly on the hardware amplifier.

In the conventional NFB scheme, the acquisition software 4, transmits suitable display control digital output to computer peripherals 8, including various forms of sensory feedback 9 to the client 1 (this completes the client loon), and suitable information to the control display 10 for display to the user 11, who uses that information to exert keyboard and mouse input 12 to the acquisition software 4, making intermittent settings and adjustments, the results of which are then displayed back to the user (completing the user loop).

In the z-score NFB scheme, the acquisition software 4 computes voltage measures and transmits those to the z-score lookup software 5, which in turn transmits corresponding z-score values back to the acquisition software 4. The acquisition software may contain a data-compression display algorithm 7, which then functions as in the conventional scheme described above. The algorithm computes one control-variable from a large number of input variables.

In the z-score NFB enhanced by the invention scheme, the z-score lookup software 5 also transmits the z-scores to the invention software 6, which performs several functions described below and in the remaining figures.

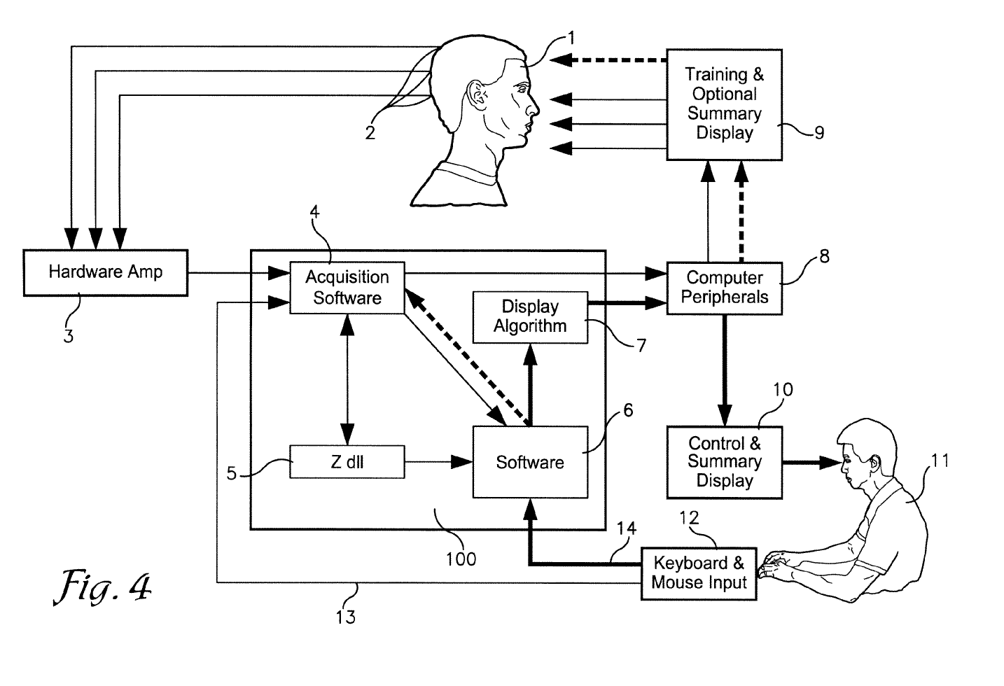

Referring to FIG. 4, in another embodiment (referred to as the “Monitor”), the present invention receives a flow of real-time z-scores from the EEG acquisition software and provides an on-screen display of summarized information. In one embodiment, the summarized information is transmitted back to the EEG acquisition software for real time use with training. The Monitor creates and populates a database with the summarized information, as well as the z-scores.

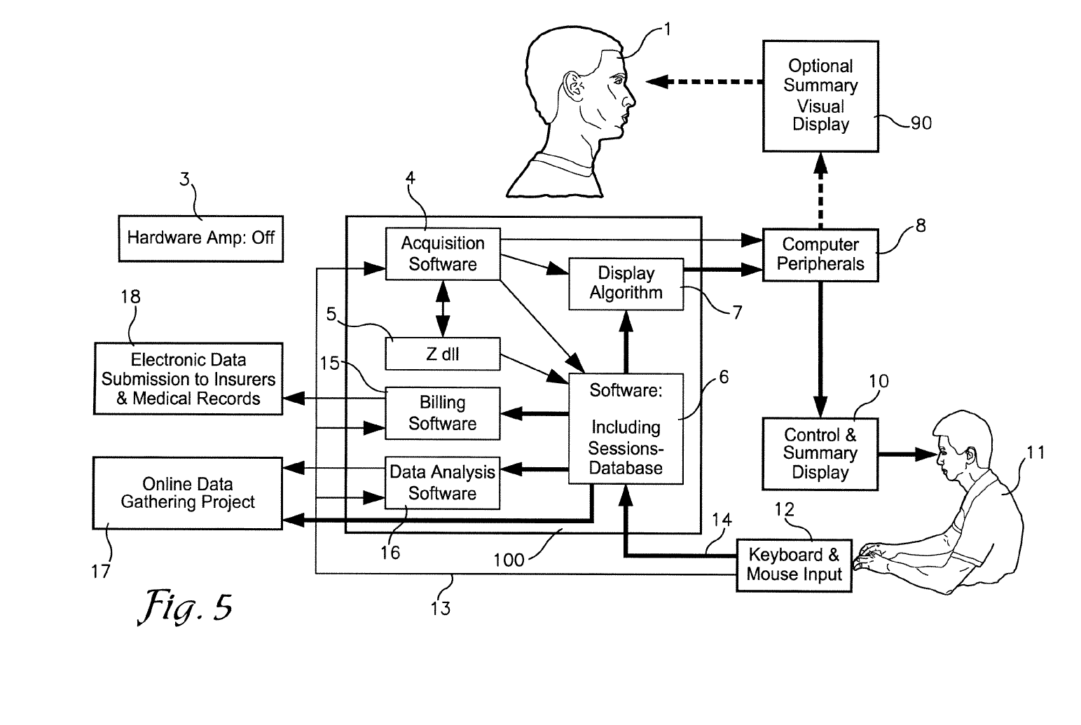

Referring to FIG. 5, in one embodiment, options allow a user to export the summarized information to third party billing software or electronic records software 18, such as a medical records system. In one embodiment, the present invention receives various training settings information from the EEG acquisition software 4 that can be saved in and correlated with the summarized information in the database.

When the Monitor output is used as a NEB training control measure, processing must occur within very narrow time constraints for the physiological training to be valid and effective. As established by manufacturer conventions and emerging professional society benchmarks, that requires at most approximately a 20 to 50 microsecond delay for the entire feedback loop, including all software components.

The Monitor employs the same functions as the Planner, but in a real-time environment. It receives continuous z-score data from the EEG acquisition software and related software applications and produces and displays the summarized information on-screen, employing the same transforms as the Planner. In one embodiment a minimum-mean-deviance training-control variable is transmitted back to the EEG acquisition software, and the measures used are stored for later use. A report is created that summarizes treatment progress on screen, suitable for printing, inclusion in electronic medical record systems, or export to third-party payer documentation. In one embodiment the Monitor facilitates the data mining of the stored data, including the z-scores stored along with the summarized information, and the training settings information discussed above. In one embodiment, the report ranks the sites or pairs by deviance. In one embodiment, the sites and pairs are displayed in the conventional order to facilitate comparison across sets of data (such as across sessions) and further summary information is computed, made possible by the use of deviance scores.

In one embodiment, the Monitor facilitates the semi-automatic collection of pre- and post-session baselines, from which it extracts within-session and cross-session (data from more than one session) progress information and due to the data structure allows measures of a client's total progress including, in one embodiment, combined measures of the impact of various protocols using multiple sites and variables. The user interface provides user control of several features of the baseline process and resulting reports.

In one embodiment, the Monitor detects suspect database compatibility based on excess z-scores, and accepts or rejects baseline data on a real-time basis. The training and baseline systems prompt for and allow the user to exclude sites or variables, based on a user-specified treatment plan derived from the Planner results, using the same auto-detection of suspect database compatibility. This functionality addresses the breach-rhythm problem.

In one embodiment, the Monitor or Trainer obtains in real-time the mean and standard deviation computed on the continuous overall-deviation scores for each site, in a brief (30-60 second) trailing window of “good data”. When new values from any site display a several-second trailing window mean which exceeds a threshold (in one embodiment, a standard deviation of 4 or more), then that several-second sample and subsequent data from that site are classified as likely to represent artifact contamination rather than reasonably pure EEG, and that suspect data-timeframe is not accumulated from any sites as “good data”. Incoming data is re-classified as acceptable when the running several-second mean of that new data is stable within 2 standard deviations of the “good data” mean. In one embodiment these standards may be adjusted by user.

For collection of baseline data, in one embodiment, the Monitor provides to the client a selectable audio-visual target, with a visual feedback of time remaining to complete baseline and overall artifact suppression. The Monitor provides the user with specific artifact monitoring and allows real-time shaping of artifact threshold(s) by user-transmitted instructions to the acquisition system, but records baseline data based on z-scores. Thus, the Monitor can be used to train the client to provide good data. The Monitor tracks both the artifact measures and the z-scores, records the baseline of good data; and pauses the z-score collection when artifact exceeds threshold. This allows a user to pause or resume for electrode correction, and marks all pauses, facilitating collection of valid z-scores during baseline. Artifact includes high impedance where impedance data is available in real-time from the EEG acquisition system. Options allow a user to collect, repeat, or skip, and provides a warning about a limited ability to provide progress data if the baseline is skipped.

In one embodiment, the Monitor calculates a simple mean-deviance and standard deviation progress report after pre-baseline and presents the results to the user. In one embodiment, termed the multi-session system (MSS), the Monitor includes a comparison to the most recent comparable dataset. In one embodiment, the Monitor provides a comparison to pre-baseline of the post-baseline findings and optionally presents a display to the client combining the two reports.

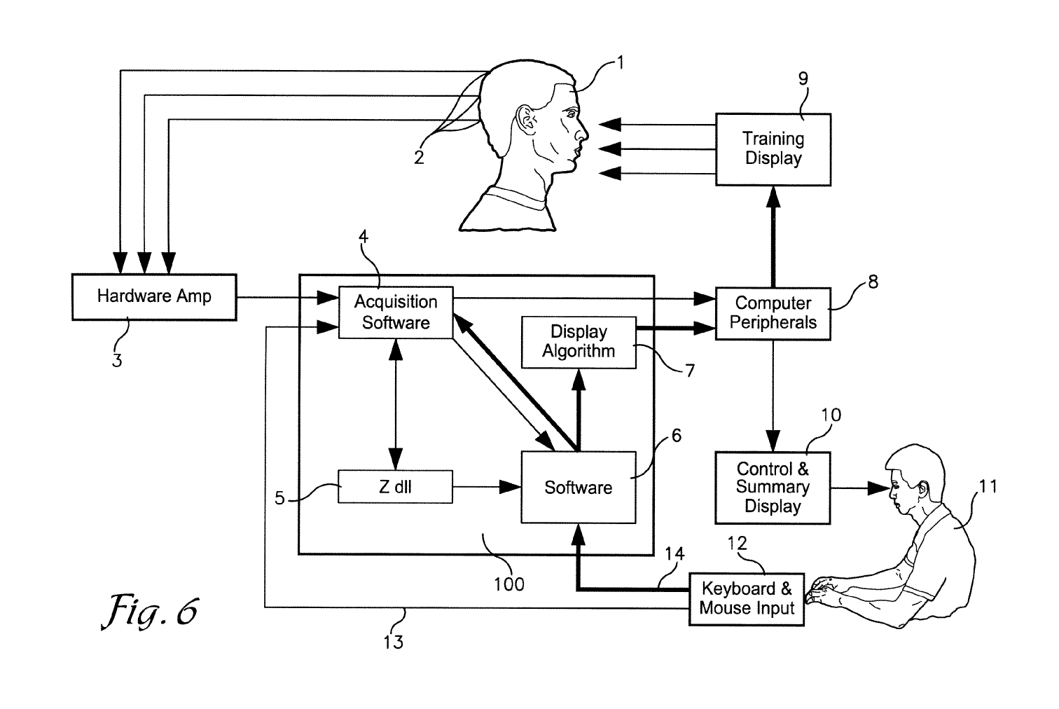

Referring to FIG. 6, in one embodiment (referred to as the “Trainer”), the present invention receives real-time z-score data employed in a feedback-training system, from which it produces a continuous monitoring report, which is displayed, stored, and can later be retrieved for additional analysis or export. The Trainer user interface allows a user to select among sites and variables at sites, by employing the same weighting rubric employed in the Planner, described above. The Trainer exports a deviance-based training-control variable and provides substantially automated regulation of certain aspects of training.

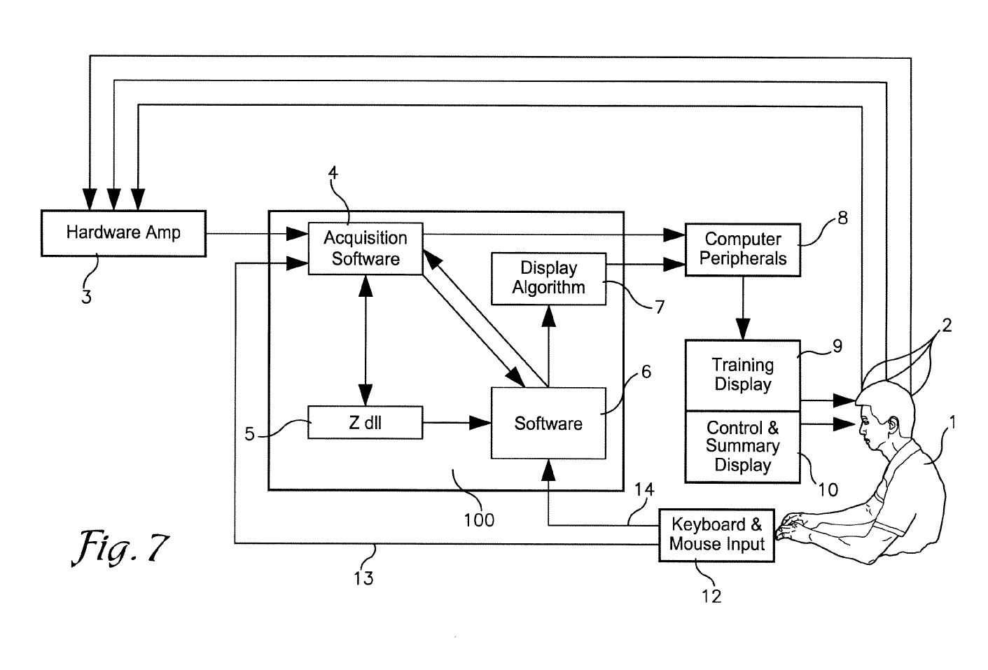

In one embodiment, the present invention 6 transmits instructions to the acquisition software 4 or other simultaneous treatment system based on routines defined or pre-selected by the user 11, which in turn exert enhanced user control over the client feedback-loop or other treatment system. Referring to FIG. 7, in one embodiment, the Trainer is a self-use device, whereby system adjustments are preset by the trainer, and a single display 9,10 is utilized by the client. In one embodiment, the Trainer is a remote-use system. In one embodiment, the Trainer includes EEG acquisition capabilities.

In one embodiment, a feedback display is provided to the client using deviance in a three dimensional topograph and features zoom-in functionality on the variables of greatest interest selected by a user and, in one embodiment, arranges information as a “brain-globe” with connections between “site-cities”. In one embodiment, the display is accessible to color-blind persons.

In one embodiment, the Trainer and Monitor each optionally import and integrate the Planner report, in order to transfer settings into the real time system. To ensure that transferred settings are based on valid data, a user is required to confirm the responses to the Planner inquiry regarding any medical history of breach in the skull. In one embodiment, to further ensure valid settings, the settings are derived from at least two independent analyses conducted with the Planner.

In one embodiment, the Trainer utilizes the adjustable multiplicative weights, described above, to allow the user to select or de-select sites, or variables and frequency-bands at selected sites, for computation of the control variable(s) used in NFB training. In one embodiment, during training the Monitor provides reports on the entire data stream or on the selected data. In one embodiment, in addition to the single outcome variable minimum-mean-deviance (MMD), the Trainer presents any sub-component mean-deviance, and transforms the result to a value ranging from 0-1, facilitating communication to output display systems.

The issue of skull breach is just as important in training as in assessment. By allowing the user to select sites, or variables and frequencies at sites, the Monitor and Trainer give the user enhanced ability to manage this issue. In one embodiment, the Trainer prompts and requires the user to indicate or deny presence of skull breach. In one embodiment, the Trainer allows creation of more than one stream of output to the acquisition software or to the display, consisting of deviance scores computed from various subsets of the data stream. This allows the acquisition system to receive from the Trainer and manipulate several real-time variables.

In one embodiment, a subset is selected of orthogonal variables which may or may not contain the full range of the underlying measure, such as EEG frequency. Operating with that subset, the absolute-value is taken of each member and a further selection is made of all subset member absolute-values that meet or exceed a chosen threshold; these cells may be termed “hits”. A count is made of the hits, and for one or more variables the hit absolute-values are averaged. If for more than one variable then the average may or may not he computed across variables. In one embodiment, the hit-count and absolute-values-averages are obtained for the site and pair subsets that exceed threshold, and the results employed in computing the MMD described below.

In one embodiment, the chosen threshold may be fixed as a level of significance such as the 95% confidence cutoff for the real-time z-score calculation (which is considerably lower than the more familiar one at [Z]=1.96). In one embodiment the chosen threshold may be set by a user. In one embodiment, the chosen threshold may be set by a user along a continuous range interactively by means of adjusting a slider-bar or other means.

In one embodiment, the Trainer trains the client to minimize the mean overall deviance, calculated using the multivariate computational formula developed for the Planner. The MMD yields one training variable, a continuous measure, for each site. Derived from the matrix of deviances provided by the Planner, the MMD is multiplied by a weight factor, either 0 for excluded values or otherwise ranging from 0.1 to 1.0.

In one embodiment, the overall mean deviance for site s {D(s)} is calculated as follows:

D(s)=k₁.powerD(s)+k₂.asymD(s)+k₃.cohD(s)+k₄.phaD(s)

(Number of non-zero k-terms in the numerator)

Where:
1. D is the deviance, calculated as [Z],
2. kᵢ is a weighting factor supplied by the user for each component of the numerator,
3. kᵢ can take the values of 0, or 0.1-1.0, with default value=1,
4. powerD(s)=mean(power D for each orthogonal frequency range), for site s, and
5. asymD(s), cohD(s), and phaD(s) are defined as follows.

For the above calculation, the deviance for each of the three connectivity variables (asymmetry, coherence and phase) is derived from the deviance for the site-pairs:
(a) First, the mean deviance across frequency bins is calculated for each of the 171 pairs, separately for asymmetry, coherence, and phase.
(b) That pair deviance is attributed to both the first and second site in each pair.
(c) Then the mean is calculated of these attributed deviance values for each site, separately for asymmetry, coherence, and phase.
(d) The resulting mean deviance values are used in the above equation.

In one embodiment, when calculating deviance for site-pairs, the overall mean deviance across power, asymmetry, coherence & phase is calculated for each pair, in a calculation that includes the weighting scheme above. For pair s1-s2 the overall mean deviance for that pair is:

D(p)=k₁.(powerD(s1)+powerD(s2))______2______+k₂.asymD(p)+k₃.cohD(p)+k₄.phaD(p)

(Number of non-zero k-terms in the numerator)

Where:
1. D is the deviance, calculated as [Z],
2. kᵢ is a weighting factor supplied by the user for each component of the numerator,
3. kᵢ can take the values of 0, or 0.1-1.0, with default value=1,
4. powerD(s)=mean (power D for each orthogonal frequency range), for site s, and
5. asymD(p), cohD(p), and phaD(p) are the mean deviance across frequency bins calculated for each of the 171 pairings in step (a) above.

In one embodiment, the deviance D is calculated as the absolute value of (z-score/chosen threshold), rather than as [Z]. That is:

D=[Z/T]

Where T is a chosen threshold.

In one embodiment, the deviance D is calculated as the square of (z-score/chosen threshold). That is:

D=(Z/T)²

Where T is a chosen threshold.

In one embodiment, the deviance D is calculated as the product of a chosen threshold and the square of (z-score/chosen threshold). That is:

D=T(Z/T)²

Where T is a chosen threshold.

In one embodiment, the value of the deviance D or of any mean of deviances calculated so as to employ a chosen threshold T, may be examined and a second threshold (such as 1.0) employed as a cutoff below which the value may be excluded from or treated differently in subsequent calculation or display. In one embodiment a count may be made of the number of deviance values which meet or exceed the chosen threshold and that count employed to define a hit count or hit rate.

In various embodiments, deviance may be calculated in MMD training one of the following three methods:
(1) MMD: Utilizing the general solution above.
(2) MMD′: Allowing the user to set bounds on a z-score range, providing a training reward as the scores approach that range, and measuring the excess deviance beyond an upper and a lower threshold. The excess, D′ is a function of [Zᵢ−Tu˜lᵢ], where Z is the z-score, and Tu & Tl are the upper and lower thresholds for a given variable. Then

If Z>Tu, then D′=[Z−Tu]

If Z<Tl, then D′=[Z−Tl]

If Tl<=Z<=Tu, then D′=0

This allows the specification of separate upper and lower thresholds for classes of variables, and if desired allows the thresholds to be set so that both are on the same side of zero. Training is toward D′=0.
(3) Providing a phenotype correction to Power z-scores and computing the Power deviance for each site so as to include a site-specific additive constant:
   powerD=Ai [Zi+ki], where Ai is the weight, Zi is the z-score, and ki is an additive correction to the z-score, for cell i in the z-score table of sites x frequency-bands.

Referring to FIG. 5, in one embodiment, the present invention provides a nineteen channel z-score NFB data-mining platform implemented as an off-line research tool as discussed above. In one embodiment, the system captures substantially all of the large data-stream available from 19 channel training and involves significant raw processing, requiring specialized computing hardware. In one embodiment, the system down-samples to lower sampling rates. The collection and processing of this data is key to defining the empirically based rules for the Trainer. Rule-driven routines improve the meta-session system (MSS) reporting of cross-session results by the Monitor, and in the Trainer use the cross-session results to flag problem protocols, recommend protocols, recommend protocol taper, recommend protocol reinforcement-schedule changes, recommend protocol reinstatement, and recommend termination of completed training. In addition, the rules rely on cross-session results to provide real-time, substantially automatic control of NFB sessions, based on user pre-approved NFB treatment meta-protocols, reducing continuous user manual-input.

In one embodiment, the Trainer facilitates shaping, the process in operant conditioning by which a user/trainer coaxes the desired response from the client being trained, so that the response may then be reinforced and strengthened. Shaping has been shown to have desirable effects during sessions. In order to shape good task response, threshold feathering is implemented by the derived rules. It is beneficial in earlier sessions, before the response is well-learned and can be sustained through the session, and in later sessions, such as for artifact that is outside the client's awareness or to help the client overcome loss of task-focus. In these instances, when reward rate drops, a change in threshold that results in increasing reward results in a surge in task-related response.

In one embodiment, the shaping rules employ a routine that is analogous to trend analysis of traded investments. The trend of the control variable is computed real time, break-out thresholds are computed based on recent history and on empirically derived expectations, and the rules are set to adjust the reward threshold based on trend analysis results. In one embodiment, one, several, or all of the reinforcement schedules recognized in the operant conditioning literature are implemented in the present invention.

All present NFB training systems continue to confound the recording of results with any changes the user makes to adjust training, such as choosing training duration and threshold levels. The Trainer is specifically designed to overcome this confound. It allows continuous z-score data collection, and artifact estimation, but separately allows changes to the feedback stream including pauses that do not interrupt data flow nor confound results.

In order to shape the desired physiological response in conventional NFB, the thresholds are manually adjusted by the user. One problem in this arrangement is that this process can require nearly continuous user involvement. The larger problem is that meeting threshold is a meaningless outcome measure in nearly every data design, but is commonly relied upon in clinical applications. The use of z-scores offers a solution to this issue. The z-level criterion (that z-score chosen by the user as “good enough”) generally is implemented as a relatively static calibration, although one of which the user must remain aware, but if the z-level criterion is used to shape response then again success is only a relative measure. In various embodiments, the present invention helps the user gather the baseline data needed for the Monitor's MSS and implement contingent reward strategies, serves as the basis for automated baseline and automated shaping, integrates discrete trials with continuous reinforcement, and implements dosing of training duration. These solutions allow the invention to provide valid and meaningful outcome measures.

In one embodiment, after obtaining a pre-training baseline measure of deviance and relying on the baseline procedure in which there is feedback of artifact-reduction only, a fourth method for calculating MMD is accomplished by calculating the difference between the baseline deviance and a deviance corresponding to the desired z-level criterion. A discrete-trials design is implemented by conducting a series of brief training trails, followed by a post-session baseline. Incremental deviance improvements are rewarded from the pre-baseline toward criterion; that is, reward is provided as the score for each short training interval approaches criterion by a fraction of the difference, and reward is withheld if there is no incremental improvement.

Although the invention has been disclosed with reference to various particular embodiments, it is understood that equivalents may be employed and substitutions made herein without departing from the scope of the invention as recited in the claims.

Having thus described the preferred embodiment of the invention, what is claimed as new and desired to be protected by Letters Patent includes the following:

1. A diagnostic system analyzing EEG data comprising:
single-site data from at least one single-site;
a computing device operable to calculate a single-site deviance for the single-site data and a single score for each of a plurality of scalp sites based on the single-site deviance; and
an output device ranking the single scores to identify sites showing abnormal EEG.

2. The system of claim 1, further comprising site-pair data from at least one site-pair, wherein the computing device is further operable to calculate a site-pair deviance for the site-pair data and the single score is calculated for each of a plurality of scalp sites based on the single-site deviance and corresponding site-pair deviance.

3. The system of claim 1, wherein the computing device is operable to export the single scores as output to EEG acquisition software.

4. The system of claim 1, wherein the single-site data is represented by at least one z-score.

5. The system of claim 1, wherein the site-pair data is represented by at least one z-score.

6. The system of claim 1, wherein deviance is calculated by taking the absolute value of the at least one z-score.

7. The system of claim 1, further comprising a deficit input for allowing an operator to identify specific sites where skull deficits exist, wherein the computing device is operable to exclude data from these sites.

8. The system of claim 1, wherein the data is static.

9. The system of claim 1, where the data is in real-time.

10. The system of claim 1, further comprising at least one cross-session report that combines input from multiple types of EEG measures.

11. The system of claim 10, wherein the EEG measures comprise Linked Ears.

12. The method as set forth in claim 10, wherein the EEG measures comprise Average Reference.

13. The method as set forth in claim 10, wherein the EEG measures comprise Laplacian.

14. The system of claim 1, wherein the corresponding site-pair deviance includes calculating deviance for each site-pair data that includes as one of the sites in the site-pair a site corresponding to the single-site for which a score is being calculated.

15. The system of claim 1, wherein the computing device is operable to export the single scores as output for integration with billing software.

16. The system of claim 1, wherein the single scores are operable to measure effects of medications.

17. The system of claim 1, wherein the single scores are operable to measure treatment response.

18. The system of claim 1, further comprising a display operable to graphically display a representation of the scores.

19. The system of claim 1, further comprising a database created and populated with information.

20. The system of claim 19, wherein information comprises the data, the single-site deviance, the site-pair deviance, and the single scores.

21. The system of claim 1, further comprising an interest input for allowing an operator to specify at least one site of interest, wherein scores from the at least one site of interest facilitate hypothesis testing.

22. The system of claim 1, further comprising a trainer output device operable to facilitate operant conditioning of a client's EEG.

23. The system of claim 1, further comprising a collection of baseline data.

24. The system of claim 23, wherein the collection of baseline data is substantially automatically collected by the computing device.

25. The system of claim 24, wherein the collection of baseline data facilitates exclusion of artifact.

26. The system of claim 24, wherein the collection of baseline data facilitates exclusion of pauses.

27. The system of claim 1, further comprising a chosen threshold deviance.

28. The system of claim 1, wherein deviance is calculated by taking the square of the at least one z-score.

29. The system of claim 1, wherein deviance is calculated by taking the square of the at least one z-score divided by a chosen threshold.

30. A method of analyzing EEG data comprising the steps of:
loading EEG single-site data;
determining a single-site deviance for the single-site data;
determining a single score for a plurality of scalp sites; and
producing a ranked report identifying sites showing abnormal EEG.

31. The method as set forth in claim 30, further including the step of loading EEG site-pair data.

32. The method as set forth in claim 31, wherein the site-pair data is represented by at least one z-score.

33. The method as set forth in claim 31, further including the step of determining site-pair deviance.

34. The method as set forth in claim 33, wherein the single score for each of a plurality of sites is determined by utilizing the single-site deviance and each site-pair deviance for each site-pair that includes as one of the sites in the site-pair a site corresponding to the single-site for which a single score is being calculated.

35. The method as set forth in claim 30, further including the step of exporting the single scores to EEG acquisition software.

36. The method as set forth in claim 30, wherein the single-site data is represented by at least one z-score.

37. The method as set forth in claim 30, wherein deviance is calculated by taking the absolute value of the at least one z-score.

38. The method as set forth in claim 30, further including the step of excluding specific sites where skull deficits exist.

39. The method as set forth in claim 30, wherein the data is static.

40. The method as set forth in claim 30, wherein the data is in real-time.

41. The method as set forth in claim 30, further including the step of producing reports that combine input from multiple types of EEG measures.

42. The method as set forth in claim 41, wherein the EEG measures comprise Linked Ears.

43. The method as set forth in claim 41, wherein the EEG measures comprise Average Reference.

44. The method as set forth in claim 41, wherein the EEG measures comprise Laplacian.

45. The method as set forth in claim 30, further including the step of allowing an operator to specify at least one site of interest to facilitate hypothesis testing.

46. The method as set forth in claim 30, further including the step of providing feedback to a client to facilitate operant conditioning.

47. The method as set forth in claim 30, further including the step of substantially automatically collecting baseline data.

48. The method as set forth in claim 30, further including the step of excluding artifact.

49. The method as set forth in claim 30, further including the step of excluding pauses.

50. The method as set forth in claim 30, further including the step of selecting a chosen threshold deviance.

51. The method as set forth in claim 30, wherein deviance is calculated by taking the square of the at least one z-score.

52. The method as set forth in claim 30, wherein deviance is calculated by taking the square of the at least one z-score divided by a chosen threshold.

53. A computer program for analyzing EEG data, with the computer program being stored on a computer-readable medium and executable by a computing device, the computer program comprising instructions for:
selecting input variables;
loading EEG single-site data;
loading EEG site-pair data;
determining a single-site deviance for the at least single-site data;
determining a site-pair deviance for the site-pair data;
determining a single score for a plurality of scalp sites from the single-site deviance and corresponding site-pair deviance; and
presenting on a display a ranked report identifying sites showing abnormal EEG.

54. The computer program of claim 53, further including instructions for identifying and excluding from the single score determination deviance scores for sites where skull deficits exist.

55. The computer program of claim 53, further including instructions for selecting a chosen threshold deviance.

56. The computer program of claim 53, further including instructions for specifying at least one site of interest.

57. The computer program of claim 53, further including instructions for requiring that a data point meet or exceed a threshold value before being included in further calculations.

58. The computer program of claim 53, further including instructions for collecting baseline data.

59. The computer program of claim 53, further including instructions for excluding artifact.

60. The computer program of claim 53, further including instructions for excluding pauses.
